(19) United States
(12) Patent Application Publication
(10) Pub. No.: US 2002/0106700 A1
(43) Pub. Date: Aug. 8, 2002

(54) Method for analyzing proteins

(76) Inventors: Robert S. Foote, Oak Ridge, TN (US); J. Michael Ramsey, Knoxville, TN (US)

Correspondence Address: George A. Nelson, Esq., Akerman Senterfitt, 222 Lakeview Avenue, Suite 400, Post Office Box 3188, West Palm Beach, FL, 33402-3188, US

(21) Appl. No.: 09/776,980

(22) Filed: Feb. 5, 2001

(51) Int. Cl. G01N 33/53; C12Q 1/37

(52) U.S. Cl. 435/7.5; 435/23

(57) ABSTRACT

A method for characterizing an individual protein contained in a complex mixture of proteins includes the steps of: providing a mixture containing different proteins; fragmenting at least one of the proteins contained in the mixture into a terminal peptide and a non-terminal peptide; separating the terminal peptide from the non-terminal peptide; and analyzing at least one chemical characteristic of the terminal peptide.

### STATEMENT AS TO FEDERALLY SPONSORED RESEARCH

This invention was made with United States Government support under grant number DE-ACO5-00OR22725 awarded by the Department of Energy. The Government has certain rights in the invention.

### CROSS REFERENCE TO RELATED APPLICATION

Not applicable.

### FIELD OF THE INVENTION

The invention relates generally to the fields of molecular biology, protein chemistry, and proteomics. More particularly, the invention relates to a method for characterizing individual proteins contained in a complex mixture of proteins.

### BACKGROUND

The human genome contains approximately 100,000 genes, of which 5,000-6,000 may be expressed in a given cell type. Celis et al., FEBS Letters (1996) 398: 129. Although DNA sequencing of the human genome has been essentially completed, determining the functions of gene products may require an effort equal to or greater than that of the Human Genome Project. Nowak, Science (1995) 270: 368. Insights into gene function are provided by their expressed protein levels in different cell types, developmental stages, organism phenotypes, disease states, responses to stimuli, etc. Measuring these levels requires the initial resolution of complex mixtures of cellular proteins. Linkage of a specific gene to its protein product may then be established by sequencing or tryptic mapping of the protein and comparison with amino acid (AA) sequences predicted from DNA sequence databases.

The conventional method for resolving cellular protein mixtures is two-dimensional polyacrylamide gel electrophoresis (2D PAGE), which separates polypeptides based on the orthogonal parameters of isoelectric point (pI) and size. The peak or “spot” capacity of this planar technique ranges from 4,000 to 10,000, depending on the available separation space or size of the slab gel. Anderson et al., Anal. Biochem. (1978) 85: 331; James, P., Biochem. Biophys. Res. Commun. (1997) 231: 1. The number of resolved polypeptides shown in published 2D PAGE databases typically ranges from about 1,000 to 3,000 per gel. Cf., Julio Celis Database; http://biobase.dk/cgi- bin/celis. Post-translational modifications, such as glycosylation or phosphorylation of specific amino acid residues, can result in multiple spots from a single polypeptide chain.

After separation by 2D PAGE, individual proteins (spots) may be extracted from the gel for further analysis. Identification strategies include peptide mapping, in which the masses of peptides produced by site-specific proteolysis are analyzed by mass spectrometry (MS) and correlated with unique mass patterns in protein databases. For example, a proteolytic enzyme such as trypsin (which cleaves polypeptides at arginine and lysine residues) can be used to fragment the extracted protein into two or more peptides. These peptides can then be analyzed by matrix assisted laser desorption ionization (MALDI)- or electrospray ionization (ESI)-mass spectrometry to determine their masses. The determined masses can then be used to screen a database to determine the AA sequences of the peptides.

In an alternative technique, AA sequence data is obtained from single peptides by tandem mass spectrometry (MS/MS), and used to screen databases for unique protein sequences. Eng et al., J. Am. Soc. Mass Spectrom. (1994) 5: 976; Yates III et al., Anal. Chem. (1995) 67: 3202; Yates III et al., Anal. Chem. (1995) 67: 1426; Figeys et al., Anal. Chem. (1996) 68: 1822. In this technique, selected peptide masses are isolated in the first stage of the spectrometer and subjected to collision-induced chemical dissociation, and the masses of the subfragments are then analyzed in the second stage to deduce the AA sequence.

Because of the time and effort required for individual protein isolation, digestion and analysis, high-throughput strategies involving direct proteolysis and peptide analysis of protein mixtures have been proposed. Yates and his colleagues have used liquid chromatography (LC) coupled with MS/MS to separate and identify unique peptide sequences from tryptic digests of protein mixtures (McCormack et al. (1997) Anal. Chem. 69(4): 767; Yates III, J. R. (1998) Journal of Mass Spectrometry 33(1): 1; Link et al. (1999) Nature Biotechnology 17(7): 676), while Smith and co-workers have proposed the use of high resolution Fourier transform ion cyclotron resonance (FTICR)-MS to identify unique peptide masses in complex protein digests. Conrads et al. (2000) Anal. Chem., 72(14): 3349. These approaches appear to be limited to small proteomes or protein subsets due to the high complexity of peptide mixtures generated by the enzymatic digestion of relatively small numbers of proteins.

Methods of simplifying the analysis of complex peptide mixtures by isolating signature peptides containing specific residues have been also been proposed for proteomic analysis. These include the derivatization of cysteines in protein mixtures with thiol-specific biotin reagents and isolation of the biotinylated peptides from tryptic digests by binding to avidin. Gygi et al. (1999) Nature Biotechnology 17(10): 994. Peptides containing histidine or glycosyl groups have also been isolated using immobilized metal affinity sorbents or lectin columns, respectively. Ji et al. (2000) J Chromatogr. B. Biomed. Sci. Appl. 745(1): 197. These methods were used with isotopic labeling and MS analysis to identify and quantitate specific proteins in complex mixtures. Database searching in these cases is limited to those peptides containing the target AA or modification. Moreover, these approaches are not necessarily comprehensive as proteins that lack the target moiety are not represented in the isolated peptide mixture.

Thus, because enzymatic fragmentation of a complex mixture of proteins creates an even more complex mixture of peptides, analyzing individual proteins contained in a complex mixture of proteins by the foregoing techniques remains cumbersome.

### SUMMARY

What has been discovered is a method for analyzing proteins, particularly complex mixtures of proteins. The method includes the steps of isolating and analyzing carboxy (C)-terminal and/or amino (N)-terminal peptides from a mixture of peptides resulting from the enzymatic digestion of a protein mixture (e.g., one obtained from a cell sample). The isolated terminal peptides can then be separated and analyzed by conventional methods such as mass spectrometry to determine their molecular masses and amino acid sequences. The resulting information can be used to identify the parent proteins by comparison with database information. In variations of the method, the peptides can also be labeled with tags such as fluorescent groups and analyzed by chromatographic and/or electrophoretic methods for comparative analysis of proteins in different cells, tissue, etc.

Each polypeptide chain in a mixture of proteins contains a single C-terminus and a single N-terminus. Thus, isolation of only the C-terminal or N-terminal peptides produced upon enzymatic digestion of the proteins in a mixture yields only a single peptide, rather than a multitude of peptides from each protein. The quantitatively isolated peptides also reflect the levels of their parent proteins in the mixture.

The invention provides several advantages over conventional techniques. First, because each polypeptide chain is represented by a single terminal peptide, the method of the invention simplifies analysis and allows quantitation of gene expression levels based on the 1:1 stoichiometry of peptide to parent polypeptide chain. Second, because the complexity of the analyzed peptide mixture is no greater than that of the original protein mixture as determined by SDS-PAGE, the method of the invention allows more efficient separations than can be achieved for whole digests or mixtures containing multiple peptides per individual protein subunit. Moreover, the peptide complexity should, in fact, be substantially lower than observed in SDS-PAGE protein analysis due to the absence of most post-translational modifications in the analyzed peptides. Third, the defined position of the peptide at the C- or N-terminus allows constrained database searching with significant improvement in the percentage of unique fragments, based on sequence or mass. Fourth, the total sample mass is substantially reduced, allowing the use of capillary or microchip separations at higher molar levels and more sensitive detection of signature peptides from low-abundance proteins. Fifth, the invention allows soluble peptides to be isolated for analysis from poorly soluble proteins and protein complexes that are difficult to analyze by conventional methods. The advantages of the invention should speed research in areas such as the investigation of gene function, the identification of disease markers, the analysis of cellular responses to drugs or environmental factors, and many other fields where characterization of proteins is important.

Accordingly, the invention features a method for characterizing an individual protein contained in a complex mixture of proteins. This method includes the steps of: providing a mixture containing a plurality of different proteins; fragmenting at least one of the proteins contained in the mixture into at least a terminal peptide and at least a non-terminal peptide; separating the terminal peptide from the non-terminal peptide; and analyzing at least one chemical characteristic of the terminal peptide. The complex mixture of proteins can be derived from a cell (such as a cell extract derived from a human cell) or tissue extract.

The step of fragmenting at least one of the proteins contained in the mixture into at least a terminal peptide and at least a non-terminal peptide can include contacting one of the proteins with a protease (or two or more different proteases) such as trypsin, endoproteinase Arg-C, endoproteinase Lys-C, or endoproteinase Glu-C.

In one variation of the method of the invention, the terminal peptide is a C-terminal peptide such as one greater than 3 amino acids in length. In this variation, at least one of the proteins can include a carboxyl group that can be blocked by amidation or esterification. The reagent used to block can be one that labels the carboxyl group with an agent detectable by mass spectrometry or fluorescence analysis. Also in this variation, the step of separating the terminal peptide from the non-terminal peptide can include contacting the terminal peptide and the non-terminal peptide with immobilized anhydrotrypsin. Where the non-terminal peptide includes a free α-carboxyl group, the step of separating the terminal peptide from the non-terminal peptide can include biotinylating the free α-carboxyl group of the non-terminal peptide and contacting the non-terminal peptide with immobilized avidin.

In another variation of the method of the invention, the terminal peptide is an N-terminal peptide such as one greater than 3 amino acids in length. Where one of the proteins includes an N-terminal amine, this variation can further include the steps of blocking the N-terminal peptide amine with an acylating agent; biotinylating the non-terminal peptide; and contacting the non-terminal peptide with immobilized avidin. The acylating agent can include a reactive group selected from the group consisting of isothiocyanate and succinimidyl ester.

In another aspect of the method of the invention, the step of analyzing at least one chemical characteristic of the terminal peptide can include subjecting the terminal peptide to mass spectrometry. This step can also include subjecting the terminal peptide to a two dimensional separation such as a chromatographic separation and an electrophorectic separation.

Where the step of analyzing at least one chemical characteristic of the terminal peptide results in a datum, the method can further include the step of screening a first database using the datum to correlate the terminal peptide with an amino acid sequence. The at least one chemical characteristic can be , e.g., the molecular weight of the terminal peptide. This method can also include the step of screening a second database using the amino acid sequence to identify a protein including the amino acid sequence. The second database can be one that includes a plurality of polynucleotide sequence and/or one that includes a plurality of polypeptide sequences.

Unless otherwise defined, all technical terms used herein have the same meaning as commonly understood by one of ordinary skill in the art to which this invention belongs. Definitions of chemical terms can be found, for example, in Hawley's Condensed Chemical Dictionary-13th Edition, R. B. Lewis, ed., John Wiley & Sons, 1997. A description of protein/peptide chemistry terms can be found in A. Fersht, Structure and Mechanism of Protein Science: A Guide to Enzyme Catalysis and Protein Folding, W. H. Freeman & Co., 1999. Definitions of molecular biology terms can be found, for example, in Rieger et al., Glossary of Genetics: Classical and Molecular, 5th edition, Springer-Verlag: New York, 1991; and Lewin, Genes V, Oxford University Press: New York, 1994.

As used herein, “protein,” “peptide,” or “polypeptide” means any peptide-linked chain of amino acids, regardless of length or post-translational modification, e.g., glycosylation or phosphorylation. Generally, the term “peptide” is used herein to refer to an amino acid chain less than about 25 amino acid residues in length, while the terms “protein” and “polypeptide” are used to refer to a larger amino acid chain. For example, a plurality of peptides are produced by proteolytic fragmentation of a protein.

By the phrase “chemical characteristic” is meant any measurable quality of a molecule. For example, molecular weight, isoelectric point, melting point, spectra produced by mass spectrometry, infrared spectrometry, nuclear magnetic resonance spectrometry, etc. are chemical characteristics.

As used herein, a “polynucleotide” means a chain of two or more nucleotides. For example, RNA and DNA are nucleic acid molecules.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic representation of a method of isolating C-terminal peptides from complex protein mixtures using anhydrotrypsin binding to remove other peptides.

FIG. 2 is a schematic representation of a method of isolating C-terminal peptides using biotin-avidin binding to remove other peptides.

FIG. 3 is a schematic representation of a method of isolating C-terminal peptides using both anhydrotrypsin and biotin-avidin binding.

FIG. 4 is a schematic representation of various methods of blocking and/or labeling protein carboxyls for use in the isolation and analysis of C-terminal peptides. EDC=1-ethyl-3-(3-dimethylaminopropyl)carbodiimide hydrochloride; RNH₂=an amine-containing label (e.g.,5-(aminomethyl)-fluorescein or tetramethylrhodamine cadaverine.

FIG. 5 is a schematic representation of a method of biotinylating peptide C-terminal carboxyl groups for use with the method of FIG. 2.

FIG. 6 is a schematic representation of a method of isolating N-terminal peptides by biotinylation and avidin removal of other peptides.

FIG. 7 is a schematic representation of a method of enriching N-terminal peptides by selectively biotinylating protein N-terminal amines.

FIG. 8 is a schematic representation of a method of selectively labeling or biotinylating the N-terminal amines of proteins, as well as blocking lysine amines. RX=an amine labeling reagent such as fluorescein isothiocyanate or an amine labeling reagent such as biotin succinimidyl ester. R′X=an amine blocking reagent such as sulfosuccinimidyl ester. *Some lysine ε-amines may react during this step.

FIG. 9 is a schematic representation of a method of biotinylating C-terminal and internal peptides for use in the method of FIG. 6. Biotin—X=an amine-reactive biotin derivative such as biotin succinimidyl ester.

FIG. 10 is a schematic representation of a method of analyzing complex protein mixtures by isolation of terminal peptides followed by their separation and analysis by mass spectrometry.

FIG. 11 is a schematic representation of a method for comparative analysis of protein samples by isolation and labeling of their terminal peptides followed by 2-dimensional separation and comparison of the separation patterns. The arrow in the two-dimensional separation indicates the position of a terminal peptide obtained only from protein sample 1.

### DETAILED DESCRIPTION

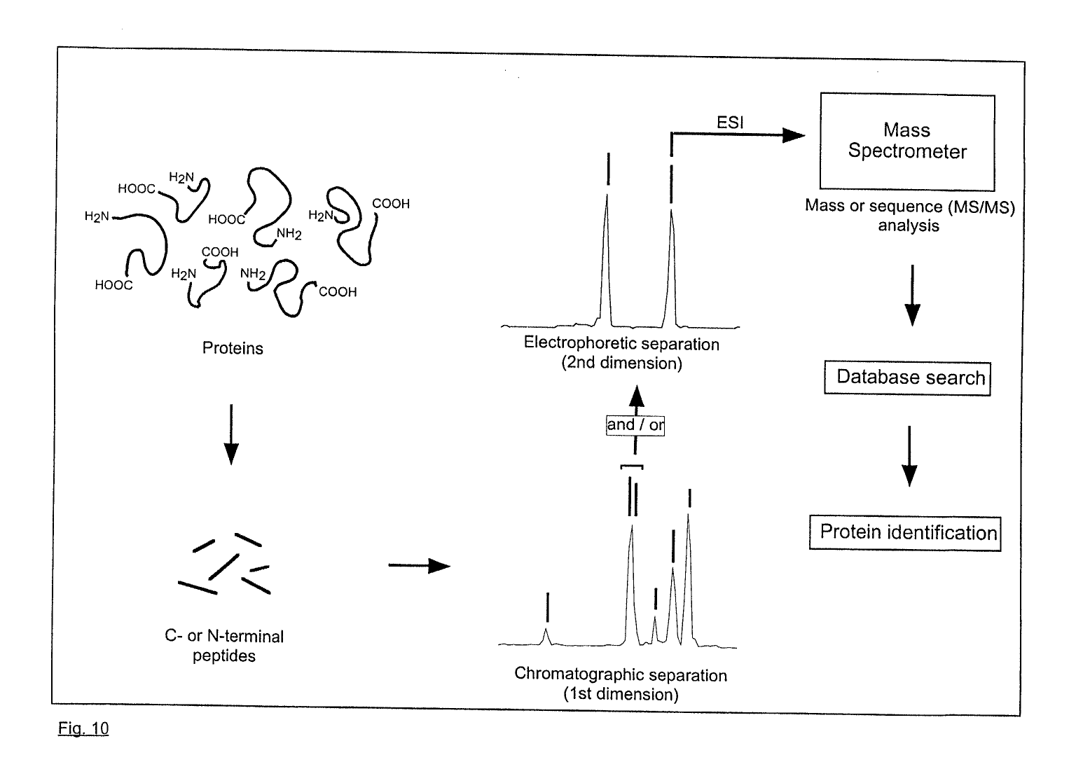

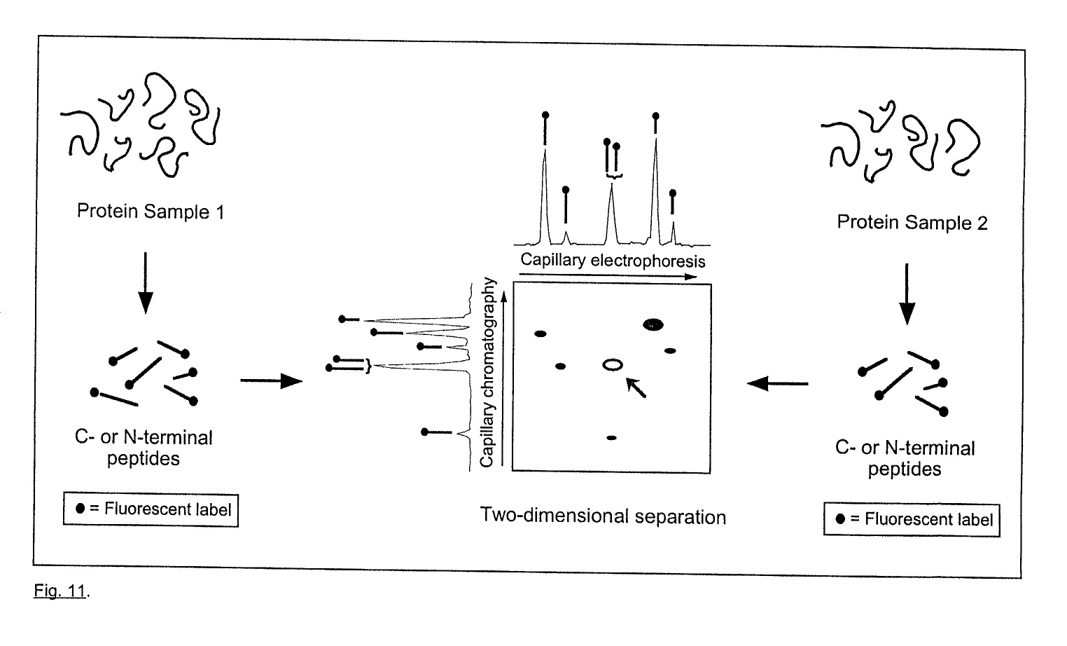

The invention provides a method for characterizing individual proteins contained in a complex mixture of proteins. The method involves enzymatically digesting the complex protein mixture into a mixture of peptides, separating the terminal peptides from the non-terminal peptides in the mixture, and then characterizing the terminal peptides by conventional methods such as by 2D column separations coupled to MS. See FIGS. 10 and 11. Information obtained from the characterization of the terminal peptides can be compared to databases including protein characterization data (e.g., gene sequence databases) to correlate a given terminal peptide with a given protein, and thus generate information about individual proteins (e.g., identity and amount present in the mixture) in the complex mixture of proteins.

The 1:1 correlation of the number of N- or C-terminal peptides to the number of parent polypeptide molecules allows straightforward quantitation of gene expression levels in the cells being assayed. In addition to the global analysis of all proteins expressed in an organism or tissue, the methods of the invention can be applied to the analysis of specific proteins or subsets of proteins for identification of disease markers, responses to drugs or environmental factors, etc.

Because the rate-limiting digestion step is carried out on all proteins simultaneously, prior to separation, this method can be performed much more efficiently than conventional methods. This approach also has the advantages of reducing sample complexity and loading mass for capillary or microchip separations and presenting the analytes in a form which can be directly analyzed on-line by MS, while preserving the information necessary to determine gene expression levels. Moreover, mixture complexity due to post-translational modifications (other than in the terminal peptide) is reduced, making separations less difficult. Fluorescent labeling and detection of the separated terminal peptides can also be used for comparative analysis of expression patterns in different cells, tissues, etc.

Peptide Length and Specificity

Short N- and C-terminal sequence tags are currently used along with protein pI and/or molecular weight to identify proteins in the SWISS-PROT database using the web-accessible program, TagIdent (http://www.expasy.ch/www/tools.html). Database analysis indicates that these sequences alone can be used for protein identification in most cases. Wilkins et al. (1998) J. Molec. Biol. 278:599. The number of possible sequences for all twenty common amino acids is 20 to the Nth power, where N is the length of the sequence in amino acids (AAs), giving numbers of 8,000 for N=3; 160,000 for N=4; 3,200,000 for N=5; and 64,000,000 for N=6. These numbers suggest that sequence tags of only 5-6 amino acids would allow unique identification of most of the ˜100,000 human proteins, assuming equal frequencies and random distributions of amino acids.

An estimate of the actual specificity of terminal sequences is provided by the analysis of Wilkins et al. (supra) who examined the uniqueness of N- and C-terminal sequence tags as a function of length and organism for proteins in the SWISS-PROT database. Their examination of 4935 human protein sequences showed that ˜80% could be uniquely identified by a 5-AA sequence at either the N- or C-terminus. The specificity was higher for prokaryotic organisms, e.g., 98% of the proteins in the database of either E. coli (3456 proteins) or B. subtilis (1889 proteins) could be uniquely identified by 5 -AA C-terminus tags.

The overall specificity for human tags is lowered by the presence of high frequency terminal sequences which are shared by proteins from the same gene family, such as those for histocompatibility antigens and immunoglobulin chains. Some polypeptides in these families vary by only a few amino acids and require complete sequencing to establish their identity. However, excluding the sequences common to members of gene families, no 5-AA C-terminal tags were found to occur in more than one protein and only one 5-AA N-terminal tag was found to occur in nonrelated proteins (an olfactory receptor-like protein as well as a family of interferon sequences). While these data are based on only ˜5% of total human proteins, along with the large numbers of possible sequences given above, they suggest that terminal sequence tags of 5 or more amino acids can be used to identify most individual proteins which are not members of gene families and to detect the presence of specific families represented by one or more members.

Generation of Terminal Peptides

Assuming approximately equal amino acid frequencies and random distribution, proteolysis at a specific type of amino acid will yield peptides with an average length of 20 AAs. However, the probability that any particular amino acid will occur within 5 residues of the terminus is approximately 23% [(1−0.95⁵)×100%]. A protease which cleaves at the terminal side of a specific amino acid would therefore produce terminal peptides of less than the desired minimum length (5 AAs) in this fraction of proteins. This problem can be addressed by using different proteases which cleave at different sites, and separate analysis of their peptide products. Cleavage at two different amino acids using different site-specific proteases would have a probability of ˜95% [(1−0.23²)×100%] of producing a terminal peptide of ≧5 AAs in at least one of the two digests, based on the above assumptions. The same probability is given for cleavage by a single enzyme if both the C- and N-termini are isolated for separate analysis, i.e., there is a 95% probability that one of the two terminal peptides will be ≧5 AAs in length. Cleavage and analysis with three different enzymes would give a probability of ˜99% [(1−0.23³)×100%] of at least one terminal fragment of ≧5 AAs. These percentages would be increased for enzymes or chemical treatments that cleave at rare sites, such as between two specific amino acids, and decreased for enzymes that cleave at multiple sites.

Trypsin normally cleaves at both arginine and lysine residues. The probability of at least one of these residues occurring within a 5-AA terminal sequence is ˜41%. However, either lysine or arginine residues can be modified so that trypsin cleavage occurs only at the unmodified amino acid, (Allen, G. (1989) Laboratory Techniques in Biochemistry and Molecular Biology. New York, Elsevier) or enzymes that cleave only at arginine (endoproteinase Arg-C) or lysine (endoproteinase Lys-C) can be used. Wilkins et al. (supra) noted a bias for lysine residues in the terminal sequences of prokaryotic proteins, which would result in decreased average length of terminal peptides with lysine-specific proteolysis. This bias was not seen in human proteins, although basic proteins such as histones contain large amounts of arginine and/or lysine and produce small tryptic fragments. Other site-specific enzymes include endoproteinase Glu-C, which cleaves on the carboxyl side of glutamic acid residues, and endoproteinase Asp-N, which cleaves on the amino side of aspartic acid. Chemical methods for cleavage at specific amino acids, including methionine and tryptophan, have also been used to prepare peptides for MS analysis. Allen, supra; Lee, T. D. and J. E. Shively (1990) Methods in Enzymology. J. A. McCloskey. 193: 361.

Isolation of Terminal Peptides

Isolation of C-terminal Peptides

Any method suitable for isolating C-terminal peptides from a mixture of terminal and non-terminal peptides resulting from the digestion of a protein mixture can be used in the invention. Two general methods have been used for isolating the C-terminal peptides of single proteins. These methods involve diagonal electrophoresis (Duggleby et al., Anal. Chem. (1975) 65: 346) and affinity chromatography on anhydrotrypsin (Kumazaki et al., Proteins (1986) 1:100), respectively. In the former approach, all carboxyl groups in a protein are first blocked by amidation or esterification, and the modified protein is then enzymatically digested with trypsin or another site-specific protease. All resulting peptides other than the C- terminal fragment will have a single ionizable carboxyl group and will electrophoretically migrate with different mobilities in buffers with pHs above (4.4) and below (2.1) the α-carboxyl pKₐ (3.0-3.2), while the mobility of the blocked C-terminal fragment is unchanged. After 2-dimensional paper electrophoresis with these buffers, only the C-terminal fragment will be located on a diagonal between two markers that have high and low mobility, respectively, and will also be insensitive to the difference in buffer pH. The C-terminal peptide can then be extracted for analysis.

In the second approach, proteins are directly digested with trypsin without prior modification, and the digest is passed through a column of immobilized anhydrotrypsin, a catalytically inactive form of trypsin which binds with high affinity to peptides having an arginine or lysine residue at the C-terminus. Because trypsin cleaves on the carboxyl side of arginine and lysine residues, all peptides other than the C-terminal fragment are bound to the column, while the C-terminal peptide passes through (unless it also has a C-terminal arginine or lysine). To isolate the C-terminal peptides of proteins having an arginine or lysine at the C-terminus an enzyme that does not cleave at these residues can be used for the digestion. In this case only the C-terminal peptides will be bound to the column at neutral pH. These can be eluted at low pH for analysis.

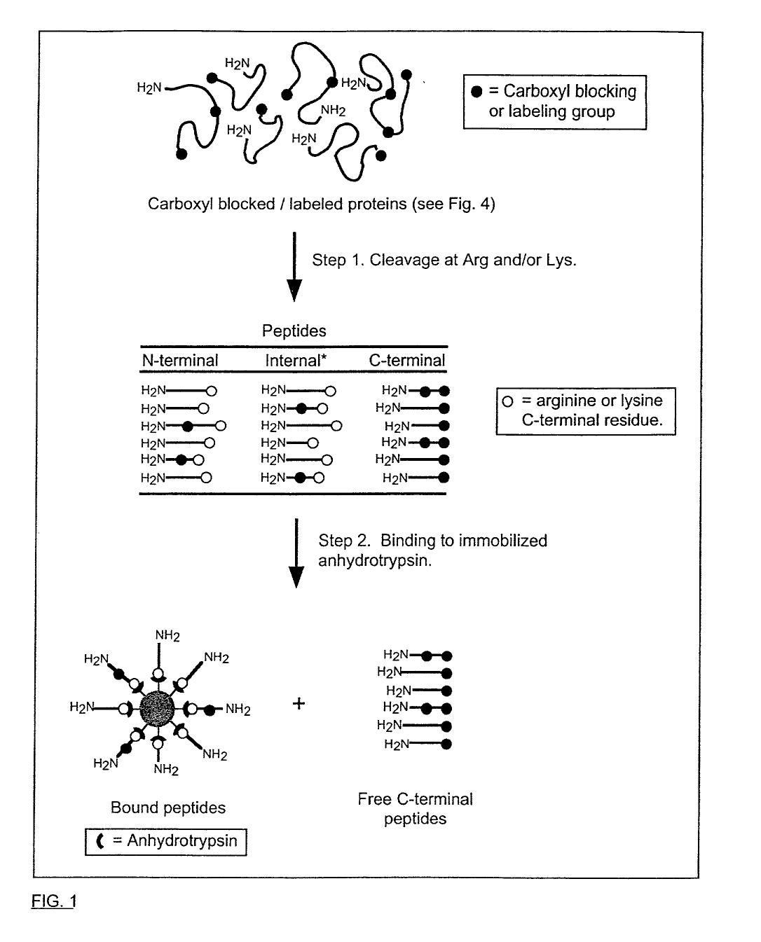

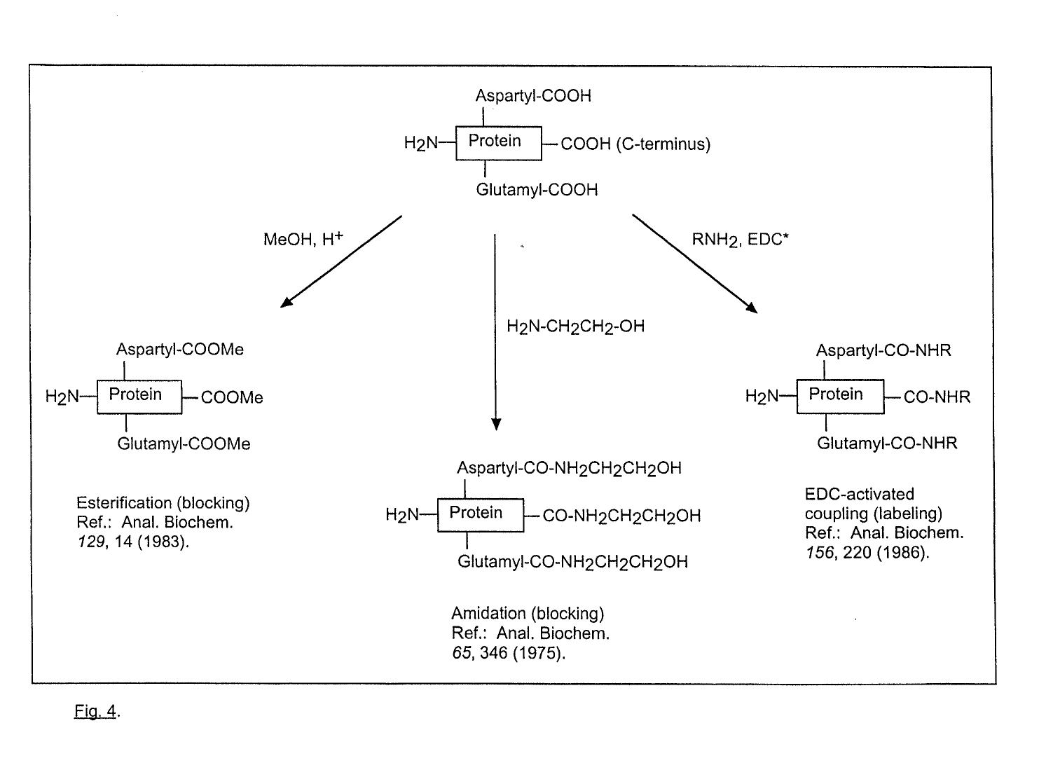

Neither of the foregoing general approaches is believed to have been applied to the isolation of C-terminal peptides from complex protein mixtures. Modified approaches for this purpose are shown in FIGS. 1-3. In each of these methods, all protein carboxyl groups are first labeled by amidation or esterification using tags for fluorescence or mass spectral analysis. For example, protein carboxyls can be labeled by coupling with hydrazines or amines using water-soluble carbodiimides. Haugland, Handbook of Fluorescent Probes and Research Chemicals (1996) p.71. Three specific examples of blocking/labeling protein carboxyl groups are shown in FIG. 4. No chemical method is believed to have been developed for specific labeling of the C-terminal carboxyl group without reaction at the side-chain carboxyls of aspartic and glutamic acid. However, terminally-labeled peptides can be distinguished from those containing labeled side-chains based on MS/MS fragmentation spectra.

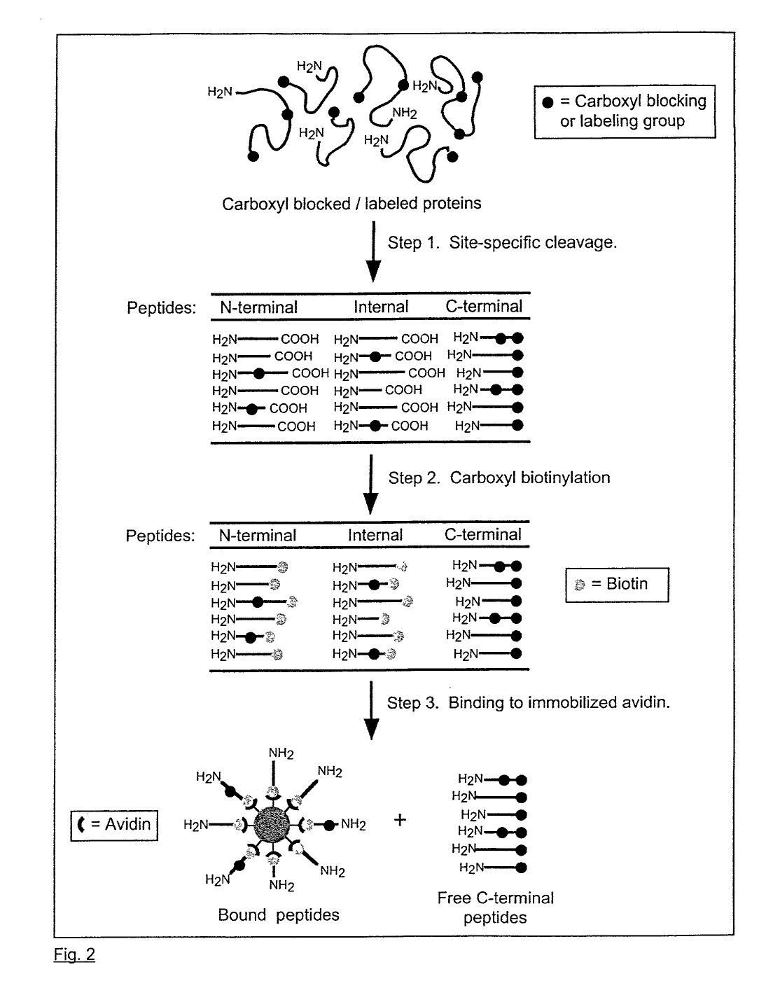

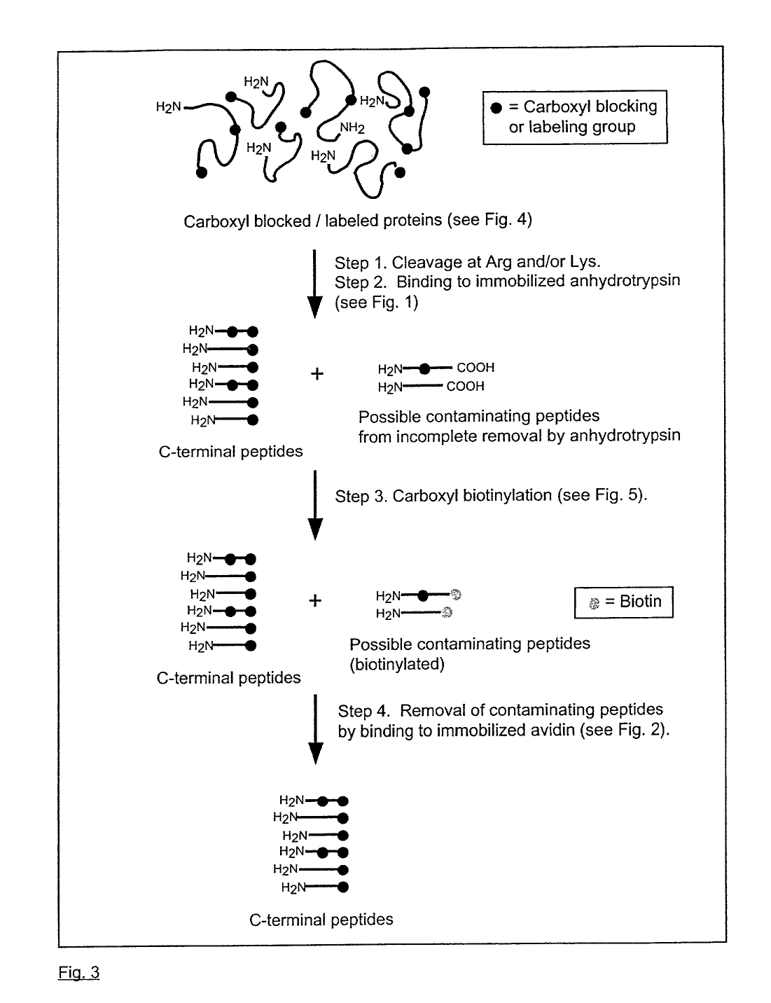

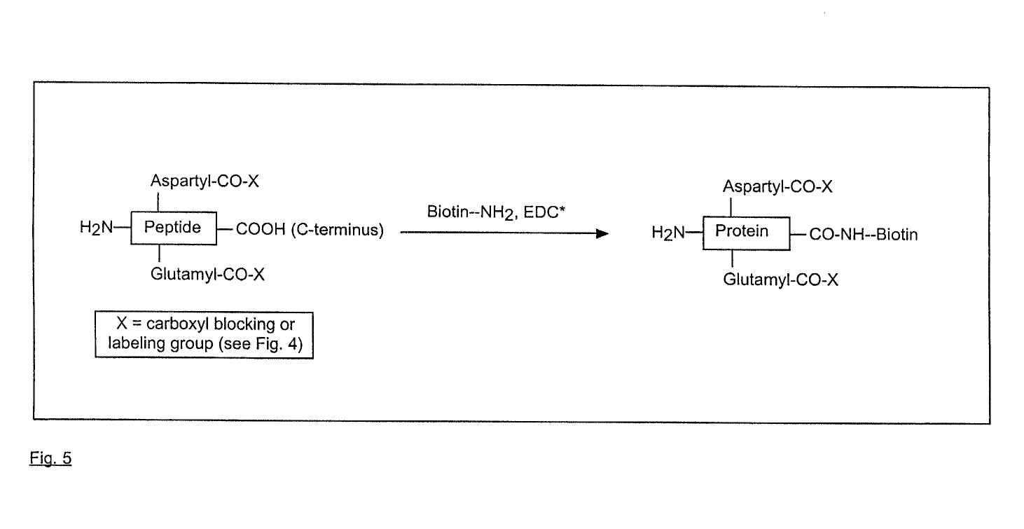

In the method illustrated in FIG. 1, after blocking/labeling the proteins are cleaved with endoproteinase Arg-C or Lys-C, and the non-terminal peptides are removed by anhydrotrypsin chromatography. Blocking/labeling of C-terminal Lys or Arg carboxyls should inhibit their binding to anhydrotrypsin so that all terminal peptides pass through the column. In the method illustrated in FIG. 2, the free α-carboxyl groups of the non-terminal peptides are reacted with biotinylating agents and the modified peptides removed by binding to immobilized avidin. An exemplary method of biotinylating peptide C-terminal carboxyl groups is illustrated in FIG. 5 in which EDC and an amine-containing biotin derivative (Biotin—NH2) such as biotin cadaverine is used. In this method, the removal of modified peptides does not depend on the presence of a C-terminal arginine or lysine, so any site-specific cleavage method resulting in a C-terminal carboxyl group could be used. Referring to FIG. 3, the biotin-avidin method of FIG. 2 could also be used after the anhydrotrypin step of the method of FIG. 1 to remove any non-terminal peptides resulting from nonspecific cleavage or inefficient binding to anhydrotrypsin. See, Kumazaki et al., Proteins (1986) 1:100.

Isolation of N-terminal Peptides

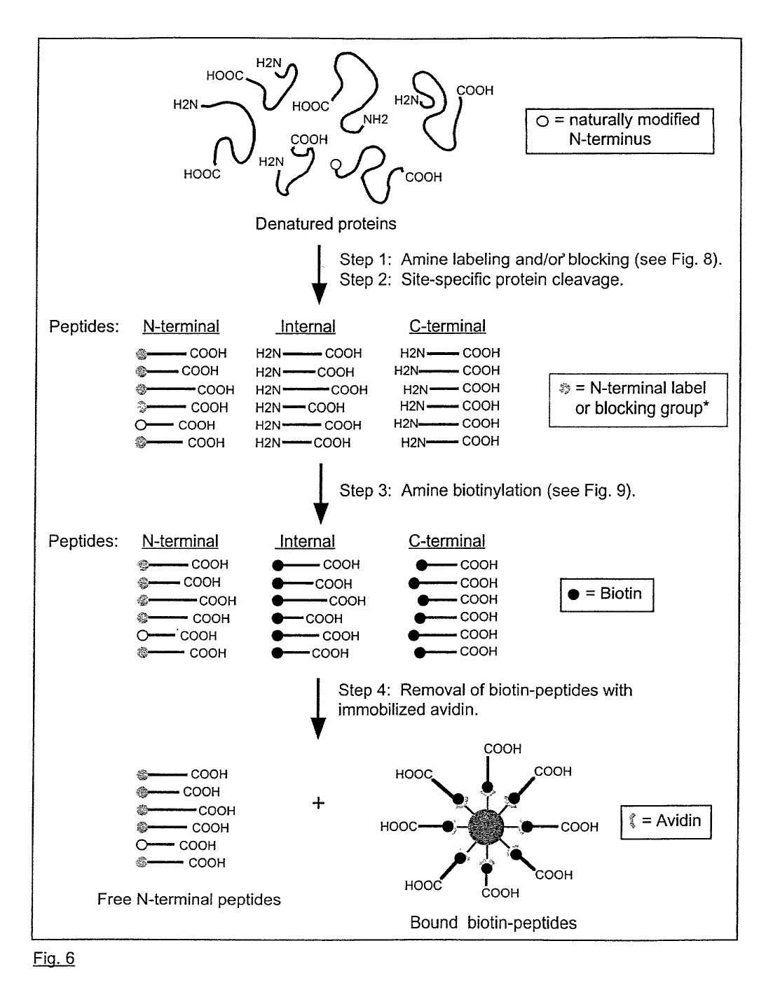

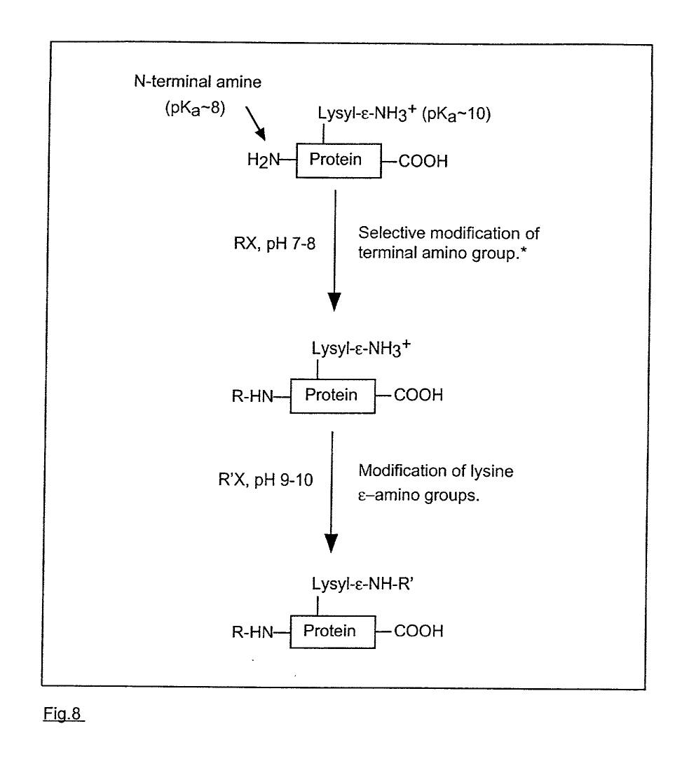

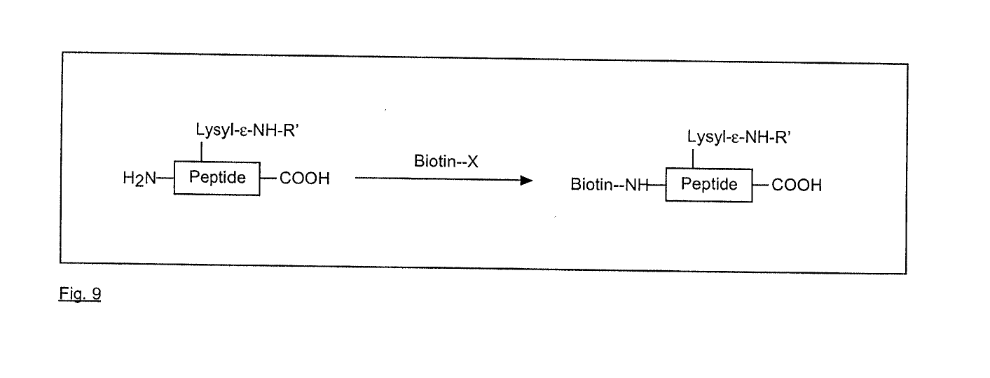

Any method suitable for isolating N-terminal peptides from a mixture of terminal and non-terminal peptides resulting from the digestion of a protein mixture can be used in the invention. The method shown schematically in FIG. 6 has been devised for use with the present invention. In this method, both the N-terminal and ε-lysyl protein amines are first blocked with an acylating reagent such as an isothiocyanate, succinimidyl ester or other amine-reactive agent designed for protein modification. See, FIG. 8 (both N-terminal amines and lysine ε-amines may be blocked with the same group by carrying out a single reaction at high pH); Allen, supra; Haugland, Handbook of Fluorescent Probes and Research Chemicals (1996) p. 8. This step can be used to label the terminal peptides with an appropriate tag for fluorescence or mass spectral analysis. The proteins are then subjected to site-specific cleavage and the N-terminal amines of all peptides, other than the N-terminal blocked peptides, are labeled with an affinity agent such as biotin (see FIG. 9) and removed by binding to an immobilized receptor ligand, e.g. avidin or streptavidin. The efficiency of removal can be monitored using an amine-reactive protein biotinylating reagent which is also fluorescently-labeled (available from Molecular Probes, Eugene, Oreg.). The N-terminal peptides from proteins that are naturally modified at the N-terminus, as well as those which are modified in the initial blocking step, are unbound and can be isolated in solution by this method. Blocking of the lysine residues can be used to prevent site-specific cleavage of this residue by trypsin or endoproteinase Lys-C, and cleavage at other sites can be performed to generate peptides. In other aspects, this method is similar to that described above for the isolation of C-terminal peptides.

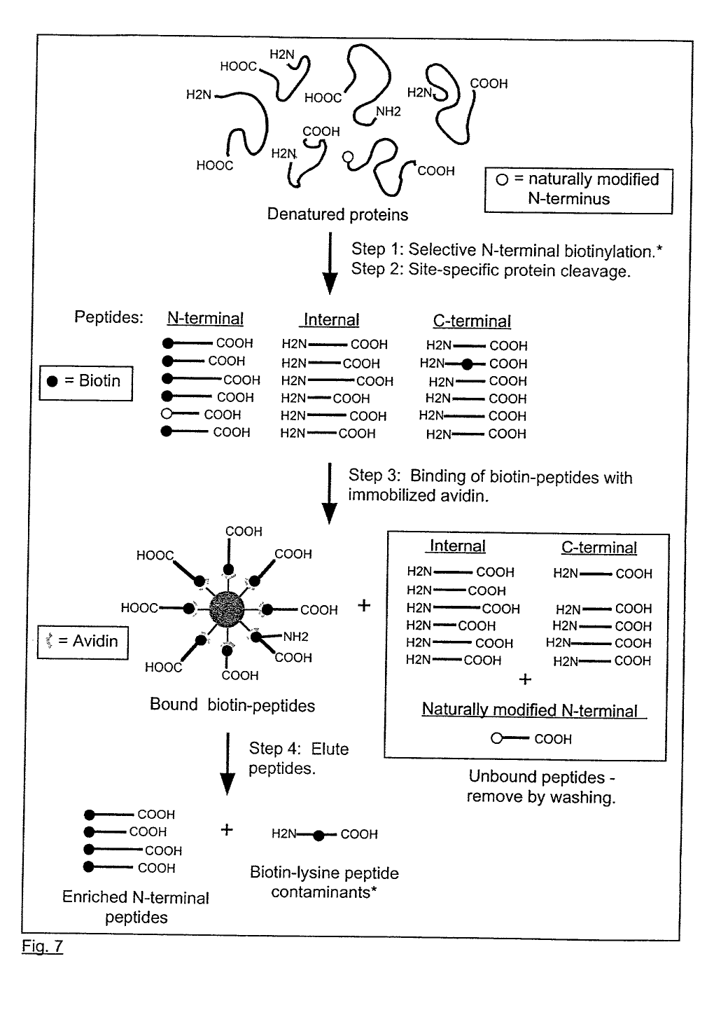

Alternative approaches for N-terminal peptide isolation are also within the invention. For example, referring to FIG. 7, the pH-dependent differences in the reactivity of the N-terminal amine (pKa 7.6-8.4) and the ε-amino group of lysine (pKₐ 9.4-10.6) can be exploited to selectively label protein terminal amines with acylating reagents at neutral pH. Selective biotinylation of the protein terminus thus allows isolation of the terminal peptide by affinity chromatography. However, naturally blocked N-terminal peptides could not be isolated by this method. The biotinylation reaction is also unlikely to be completely selective for the N-terminal amine and low levels of reaction with lysine could result in the presence of some non-terminal peptides in the isolated mixture. However, the N-terminal and ε-lysyl modified peptides can generally be distinguished by MS/MS analysis.

Characterization of Isolated Peptides

The resolution of complex terminal peptide mixtures can be performed by methods similar to those developed for complex tryptic digests. These methods generally require a combination of separation steps based on size, charge and/or polarity. For example, referring to FIG. 10, isolated N- or C-terminal peptides are first subjected to two dimensional separation (e.g., chromatographic separation in the first dimension and electrophorectic separation in the second dimension) and then mass spectrometry. The data thus obtained can be used to search databases to identify proteins having terminal peptide portions with the same characteristics as the isolated terminal peptides.

The foregoing process is amenable to automation. Automated methods combining interfaced chromatographic and electrophoretic steps have been used to resolve complex tryptic mixtures with a peak capacity of ˜3,000. Moore et al., Methods in Enzymology (1996) 207:401. Two-dimensional microchip separations have been performed with similar peak capacity. These methods can be interfaced with MS via ESI to add a third dimension to the separation, with a multiplicative increase in peak capacity, as well as for mass and sequence analysis of the separated fragments. Methods for tandem MS analysis of peptides and the correlation of spectra with sequence databases have also been automated. Figeys et al., Anal. Chem. (1995) 65: 346.

Proteins vary widely in concentration in cellular extracts and inefficiencies in the isolation of terminal peptides by the methods described above could result in a background of non-terminal fragments from which it would be difficult to distinguish the terminal peptides of low-abundance proteins. End-labeling of the proteins prior to cleavage, with tags that can be detected optically or by mass spectrometry may be used to extend the analysis to low abundance proteins. Terminally labeled peptides could be identified by tandem mass spectrometry, even in the presence of non-terminal peptides containing labeled side chains, based on altered masses of the fragment ions produced by collision-induced dissociation (CID). Naturally modified peptide residues can also be identified by this method. Yates III et al., Anal. Chem. (1995) 67: 1426. In addition, tandem MS could be used with microseparation techniques for proteome analysis based on the large reduction of sample mass achieved by separating peptide tags rather than whole protein chains.

Incomplete site-specific cleavage or non-specific cleavage could also result in low levels of terminal fragments of different lengths from the same polypeptide chain. However, so long as a unique terminal sequence is included in the spurious fragments, MS sequence analysis should reveal its presence as well as the uncleaved (or incorrectly cleaved) site, and thus identify these fragments as belonging to the same parent polypeptide.

Once peptide sequences have been identified and their elution patterns established for a given 2D separation method, additional analyses of similar samples might be performed using only elution time and parent mass for identification. Other analytical methods, such as fluorescence labeling and detection of peptides could be used to compare elution patterns of different samples and identify variations in expressed protein levels. For example, referring to FIG. 11, terminal peptides conjugated with a detectable label (e.g., a fluorescent label) are isolated from both a first protein sample and a second protein sample. Both these samples are characterized by two dimensional separation (e.g., by capillary chromatography and capillary electrophoresis). The data obtained from the first sample can then be compared to the second sample, so that the relative expression of specific proteins in the two samples can be compared.

As a more specific example, DNA repair enzymes of known structure can be induced in E. coli cells by treatment with chemical mutagens and increases in the level of the predicted terminal peptide masses could be measured by 2D separation and MS. The separation of fluorescently labeled peptides could be monitored by laser-induced fluorescence to measure variations in peptide levels. By fluorescence monitoring upstream from the ESI-MS interface, peptides showing variation could be selected for on-line MS analysis. The enzymes can be independently quantitated by conventional biochemical assays to evaluate the sensitivity and accuracy of the methods. These methods can also be extended to extracts of any other cells (e.g., cultured human cells, cells obtained from tissue samples) or extracts of tissues (e.g., fluids or extracellular material obtained from a human subject).

Other Embodiments

This description has been by way of example of how the methods of invention can be made and carried out. Those of ordinary skill in the art will recognize that various details may be modified in arriving at the other detailed embodiments, and that many of these embodiments will come within the scope of the invention.

Therefore, to apprise the public of the scope of the invention and the embodiments covered by the invention, the following claims are made.

What is claimed is:

1. A method for characterizing an individual protein contained in a complex mixture of proteins, the method comprising the steps of:
(A) providing a mixture containing a plurality of different proteins;
(B) fragmenting at least one of the proteins contained in the mixture into at least a terminal peptide and at least a non-terminal peptide;
(C) separating the terminal peptide from the non-terminal peptide; and
(D) analyzing at least one chemical characteristic of the terminal peptide.

2. The method of claim 1, wherein the complex mixture of proteins is derived from a cell or tissue extract.

3. The method of claim 2, wherein the cell extract is derived from a human cell.

4. The method of claim 1, wherein the step (B) of fragmenting at least one of the proteins contained in the mixture into at least a terminal peptide and at least a non-terminal peptide comprises contacting the at least one of the proteins with a protease.

5. The method of claim 4, wherein the protease is selected from the group consisting of: trypsin, endoproteinase Arg-C, endoproteinase Lys-C, and endoproteinase Glu-C.

6. The method of claim 4, wherein the step (B) of fragmenting at least one of the proteins contained in the mixture into at least a terminal peptide and at least a non-terminal peptide comprises contacting the at least one of the proteins with at least two different proteases.

7. The method of claim 4, wherein the step (B) of fragmenting at least one of the proteins contained in the mixture into at least a terminal peptide and at least a non-terminal peptide comprises contacting the at least one of the proteins with at least three different proteases.

8. The method of claim 1, wherein the terminal peptide is a C-terminal peptide.

9. The method of claim 8, wherein the C-terminal peptide is greater than 3 amino acids in length.

10. The method of claim 8, wherein the C-terminal peptide is greater than 4 amino acids in length.

11. The method of claim 8, wherein the C-terminal peptide is greater than 5 amino acids in length.

12. The method of claim 8, wherein the at least one of the proteins comprises a carboxyl group, and the method further comprises the step of blocking the carboxyl group by amidation or esterification.

13. The method of claim 12, wherein the step of blocking the carboxyl group by amidation or esterification labels the carboxyl group with an agent detectable by mass spectrometry or fluorescence analysis.

14. The method of claim 8, wherein the step (C) of separating the terminal peptide from the non-terminal peptide comprises contacting the terminal peptide and the non-terminal peptide with immobilized anhydrotrypsin.

15. The method of claim 8, wherein the non-terminal peptide comprises a free α-carboxyl group and the step (C) of separating the terminal peptide from the non-terminal peptide comprises biotinylating the free 60 -carboxyl group of the non-terminal peptide and contacting the non-terminal peptide with immobilized avidin.

16. The method of claim 1, wherein the terminal peptide is an N-terminal peptide.

17. The method of claim 16, wherein the N-terminal peptide is greater than 3 amino acids in length.

18. The method of claim 16, wherein the N-terminal peptide is greater than 4 amino acids in length.

19. The method of claim 16, wherein the N-terminal peptide is greater than 5 amino acids in length.

20. The method of claim 16, wherein the at least one of the proteins comprises an N-terminal amine, and the method further comprises the steps of blocking the N-terminal peptide amine with an acylating agent; biotinylating the non-terminal peptide; and contacting the non-terminal peptide with immobilized avidin.

21. The method of claim 20, wherein the acylating agent comprises a reactive group selected from the group consisting of isothiocyanate and succinimidyl ester.

22. The method of claim 1, wherein the step (D) of analyzing at least one chemical characteristic of the terminal peptide comprises subjecting the terminal peptide to mass spectrometry.

23. The method of claim 1, wherein the step of analyzing at least one chemical characteristic of the terminal peptide comprises subjecting the terminal peptide to a two dimensional separation.

24. The method of claim 23, wherein the two dimensional separation comprises a chromatographic separation and an electrophorectic separation.

25. The method of claim 1, wherein the step of analyzing at least one chemical characteristic of the terminal peptide results in a datum, and the method further comprises the step (E) of screening a first database using the datum to correlate the terminal peptide with an amino acid sequence.

26. The method of claim 25, wherein the at least one chemical characteristic is the molecular weight of the terminal peptide.

27. The method of claim 25, further comprising the step (F) of screening a second database using the amino acid sequence to identify a protein comprising the amino acid sequence.

28. The method of claim 27, wherein the second database comprises a plurality of polynucleotide sequences.

29. The method of claim 27, wherein the second database comprises a plurality of polypeptide sequences.
